(19) United States
(12) Patent Application Publication
(10) Pub. No.: US 2015/0342853 A1
(43) Pub. Date: Dec. 3, 2015

(54) Topical Lightening Composition and Methods of Use Thereof

(71) Applicant: Avon Products, Inc., Suffern, NY (US)

(72) Inventor: Uma Santhanam, Tenafly, NJ (US)

(21) Appl. No.: 14/722,427

(22) Filed: May 27, 2015

Related U.S. Application Data
(60) Provisional application No. 62/006,467, filed on Jun. 2, 2014.

(51) Int. Cl. A61K 8/66; A61K 8/97; A61K 8/34; A61K 8/60; A61K 8/46; A61Q 19/02; A61K 8/49

(52) U.S. Cl. CPC A61K 8/66; A61Q 19/02; A61K 8/97; A61K 8/4926; A61K 8/60; A61K 8/46; A61K 8/347; A61K 2800/782; A61K 2800/592

(57) ABSTRACT

Embodiments of the invention are generally directed to compositions comprising glucose oxidase containing botanical extracts, and in particular extracts of Fabiana imbricate. The compositions are useful for reducing pigmentation in the skin. The composition may further include other depigmenting agents such as nicotinamide and its melanasome transfer-inhibiting derivatives, 3,3′-thiodipropanoic acid and its tyrosinase-inhibiting derivatives, or resorcinol and its tyrosinase-inhibiting derivatives, in a cosmetically acceptable vehicle.

### CROSS-REFERENCE TO RELATED APPLICATIONS

This application claims priority to U.S. Provisional Patent Application Ser. No. 62/006,467, filed on Jun. 2, 2014. The entirety of the aforementioned application is herein incorporated by reference for all purposes.

### FIELD OF INVENTION

The invention relates generally to cosmetic and dermatological compositions and methods for reducing pigmentation in the skin. The compositions of the invention comprise the enzyme glucose oxidase, optionally in combination with a melanosome transfer inhibitor, tyrosinase inhibitor, or other agent that reduces pigmentation in the skin. More particularly, the compositions of the invention comprise a glucose oxidase containing botanical extract, for example, from the plant Fabiana imbricate. The compositions may be applied topically to skin to reduce pigmentation in the area of application.

### BACKGROUND

Several skin conditions are associated with the overproduction or unwanted production of melanin the skin, including age spots, freckles, and liver spots. The synthesis of melanin occurs in melanocyte cells in the skin and is a complex process involving several biochemical pathways. Some skin lighteners or depigmenting agents, such as hydroquinone and kojic acid, act as inhibitors of tyrosinase, an enzyme that has its catalytically active domain within organelles known as melanosomes. Tyrosinase converts phenols, including tyrosine, to ortho-quinones which are subsequently converted to melanin within the melanosomes. Other skin lighteners, such as plasminogen-activated receptor, act by disrupting the transfer of the melanosomes from melanocytes to the keratinocytes where melanin is deposited.

While skin lighteners such as hydroquinone and kojic acid have found some utility in cosmetic and dermatological products, there remains a continuing need for products that effectively reduce pigmentation of skin. It is therefore an object of the invention to provide compositions and methods for reducing pigmentation in human skin, including, for example, treatment of hyperpigmentation, unwanted pigmentation, age spots, liver spots, freckles, and the like.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### SUMMARY OF DISCLOSURE

In accordance with the foregoing objectives and other, the invention provides compositions and methods for reducing pigmentation in human skin. The compositions and methods may treat hyperpigmentation conditions, including those associated with UV damage and chronological aging, including without limitation treating, ameliorating, diminishing the appearance of, or preventing age spots, liver spots, freckles, mottled and discrete pigmentation, melisma and the like. The compositions and methods are also useful for reducing otherwise unwanted pigmentation, including overall lightening of the skin.

In one aspect of the invention, a method for reducing pigmentation in human skin is provided comprising topically applying the enzyme glucose oxidase to skin. An amount of glucose may also be topically applied to the skin prior to, after, or concurrently with the application of glucose oxidase, to serve as a substrate for the enzyme and to yield hydrogen peroxide through the oxidation of glucose. Without wishing to be bound by any particular theory, it is believed that the hydrogen peroxide generated by the reaction of glucose oxidase and glucose acts on melanin in the skin to reduce or diminish the appearance thereof.

Various non-limiting embodiments of the invention are described below. It will be understood that throughout these embodiments, the term “reducing pigmentation” includes reducing, lessening, or diminishing the appearance of hyperpigmentation, including freckles, sun spots, age spots, etc., as well as lightening skin that may or may not be affected by hyperpigmentation. Reference to human skin in each of these embodiments, includes without limitation, skin of the face, neck, arms, hands, legs, etc.

In one embodiment, a method is provided for reducing pigmentation in human skin (e.g., skin of the face) comprising topically applying to said skin a botanical extract (e.g., an aqueous, hydroalcoholic, supercritical or liquid CO₂, polar protic organic, polar aprotic organic, or non-polar aprotic organic extract) of Fabiana imbricata (e.g., the whole plant, or portion thereof, including stems, leaves, nectar and/or flower petals) comprising glucose oxidase enzyme. The extract will typically comprise at least about 1,000 units/g, or at least about 5,000 units/g, or at least about 10,000 units/g, or at least about 15,000 units/g of glucose oxidase enzyme (and up to about 100,000 units/g or even 300,000 units/g or more), including a typical range of about 15,000 units/g to about 35,000 units/g.

In another embodiment, a method is provided for reducing pigmentation in human skin (e.g., skin of the face) comprising topically applying to said skin glucose and glucose oxidase enzyme. The glucose and glucose oxidase enzyme may be applied together (e.g., simultaneously, as part of the same composition) or serially (e.g., as part of two distinct compositions), but typically contemporaneous (e.g., within 5 minutes) with one another. The enzyme may be provided from a botanical extract comprising at least about 1,000 units/g, or at least about 5,000 units/g, or at least about 10,000 units/g, or at least about 15,000 units/g of glucose oxidase enzyme (and up to about 100,000 units/g or even 300,000 units/g or more), including a typical range of about 15,000 units/g to about 35,000 units/g. The botanical extract may be of the Fabiana imbricata plant, including the nectar, whole plant, stems, leaves, and flower petals (though typically from the flower petals or from the stems and leaves). In one embodiment a botanical extract is prepared from the stems and leaves of the Fabiana imbricata plant. The extract may be an aqueous, hydroalcoholic, supercritical or liquid CO₂, polar protic organic, polar aprotic organic, or non-polar aprotic organic extract. Typically, the extract is aqueous or hydroalcoholic. In addition to the glucose and glucose oxidase enzyme, a melanosome transfer inhibitor, tyrosinase inhibitor, or other agent that reduces pigmentation in the skin may also be applied. In one embodiment, a tyrosinase inhibitor (e.g., 3,3′-thiodipropionic acid, and/or a salt and/or aliphatic C₁₋₂₀ or C₄₋₁₆ alkyl ester thereof, such as dilauryl thiodipropionate; or resorcinol or a derivative thereof, e.g., a C₁₋₂₀ alkyl derivative, such as a resorcinol having a C₁₋₂₀ or C₂₋₁₀ or C₄₋₈ alkyl group at the C-4 position, including n-butyl resorcinol and n-hexyl resorcinol) is applied prior to, after, or concurrently with the glucose oxidase enzyme. In some embodiments, niacinamide is applied prior to, after, or concurrently with the glucose oxidase enzyme.

In another embodiment, a method is provided for reducing pigmentation in human skin (e.g., skin of the face) comprising topically applying to said skin (e.g., in an amount from about 0.001 to about 100 mg/cm², more typically from about 0.01 to about 20 mg/cm², or from about 0.1 to about 10 mg/cm²) a composition comprising:
(i) an amount (e.g., from about 0.0001% to about 10% by weight, or from about 0.001% to about 5% by weight, or from about 0.01% to about 1% by weight) of glucose;
(ii) an amount (e.g., from about 0.00001% to about 10% by weight, or from about 0.0001% to about 1% by weight, or from about 0.005% to about 0.1% by weight) of a botanical extract (e.g., of Fabiana imbricate plant, typically from the stems and leaves) comprising glucose oxidase enzyme sufficient to provide at least about 0.01 activity units/g (or at least about 0.05 activity units/g, at least about 0.1 activity units/g, at least about 0.15 activity units/g) of said composition; and
(iii) a topically acceptable vehicle (i.e., non-toxic and generally regarded as safe for application to human integuments);

for a time sufficient to achieve a reduction in pigmentation in the area of application (e.g., application at least one daily for at least one week, at least two weeks, at least four weeks, or at least eight weeks). In some embodiments, the compositions are applied daily to achieve and/or maintain a reduction in pigmentation in the skin. The composition may also comprise an effective amount (e.g., from about 0.01% to about 10% by weight) of a tyrosinase inhibitor (e.g., 3,3′-thiodipropionic acid, and/or a salt and/or an aliphatic C₁₋₂₀ alkyl ester thereof such as dilauryl thiodipropionate; resorcinol or a derivative thereof, e.g., a C₁₋₂₀ alkyl derivative, such as a resorcinol having a C₁₋₂₀ or C₂₋₁₀ or C₄₋₈ alkyl group at the C-4 position, including n-butyl resorcinol and n-hexyl resorcinol). In some embodiments, the composition comprises from about 0.01% to about 5% by weight 3,3′-thiodipropionic acid and/or from about 0.01% to about 5% by weight dilauryl 3,3′-thiodipropionate. In some embodiments, the composition comprises from about 0.01% to about 10% by weight catechol, resorcinol, or hydroquinone, or a derivative thereof, including without limitation, a derivative having a C₁₋₂₀ or C₂₋₁₀ or C₄₋₈ branched, straight chain, or cyclic alkyl, alkenyl, alkynyl, aryl-alkyl, or alkyl aryl groups attached to the phenyl ring of catechol, resorcinol, or hydroquinone (e.g., at the C-2, C-4, C-5 or C-6 position of resorcinol), with particular mention being made of resorcinol having a C₁₋₂₀ or C₂₋₁₀ or C₄₋₈ alkyl group at the C-4 position, including n-butyl resorcinol and n-hexyl resorcinol. In some embodiments, the composition comprises an effective amount (e.g., from about 0.01% to about 10% by weight) of a melanosome-transfer inhibitor (e.g., niacinamide or salt thereof, or melanosome-transfer inhibiting derivative thereof).

In another embodiment, a method is provided for reducing pigmentation in human skin (e.g., skin of the face) comprising topically applying to said skin (e.g., in an amount from about 0.001 to about 100 mg/cm², more typically from about 0.01 to about 20 mg/cm², or from about 0.1 to about 10 mg/cm²) a composition comprising:
(i) an amount (e.g., from about 0.00001% to about 10% by weight, or from about 0.0001% to about 1% by weight, or from about 0.005% to about 0.1% by weight) of a botanical extract (e.g., of Fabiana imbricate plant, typically from the stems and leaves) comprising glucose oxidase enzyme sufficient to provide at least about 0.01 activity units/g (or at least about 0.05 activity units/g, at least about 0.1 activity units/g, at least about 0.15 activity units/g) of said composition;
(ii) from about 0.01% to about 10% by weight of a tyrosinase inhibitor, for example, 3,3′-thiodipropionic acid (TDPA), and/or a salt and/or aliphatic C₁₋₂₀ alkyl ester thereof (e.g., dilauryl 3,3 ‘-thiodipropionate), including embodiments wherein the composition comprises from about 0.01% to about 5% by weight 3,3’-thiodipropionic acid and/or from about 0.01% to about 5% by weight dilauryl 3,3′-thiodipropionate; or from about 0.01% to about 10% by weight of tyrosinase inhibitor comprising resorcinol having a C₁₋₂₀ or C₂₋₁₀ or C₄₋₈ alkyl group at the C-4 position, including n-butyl resorcinol and n-hexyl resorcinol (optionally, in combination with TDPA or its derivatives); and
(iii) a topically acceptable vehicle;

for a time sufficient to achieve a reduction in pigmentation in the area of application (e.g., application at least one daily for at least one week, at least two weeks, at least four weeks, or at least eight weeks). In some embodiments, the compositions are applied daily to achieve and/or maintain a reduction in pigmentation in the skin. The composition may also comprise an effective amount (e.g., from about 0.01% to about 10% by weight) of a melanosome-transfer inhibitor (e.g., niacinamide or salt thereof, or melanosome-transfer inhibiting derivative thereof). The composition may further include from about 0.0001% to about 10% by weight (or from about 0.001% to about 5% by weight, or from about 0.01% to about 1% by weight) glucose to serve as a substrate for the glucose oxidase enzyme.

Methods for reducing hyperpigmentation in human skin (e.g., sun spots, freckles, age spots, etc.) are also provided comprising topically applying glucose oxidase enzyme to an area of skin affected by hyperpigmentation (i.e., applying the glucose oxidase directly to a localized area of hyperpigmentation such as an age spot, etc.). The glucose oxidase may be provided in the form of a botanical extract (e.g., of Fabiana imbricate plant, typically from the stems and leaves) comprising an amount of glucose oxidase enzyme (e.g., at least about 1,000 units/g, or at least about 5,000 units/g, or at least about 10,000 units/g, or from about 15,000-35,000 units/g up to about 100,000 units/g or about 300,000 units/g or more) sufficient to provide at least about 0.01 activity units per gram of said composition. The composition is applied for a time sufficient to achieve a reduction in hyperpigmentation in the area of application (e.g., application at least one daily for at least one week, at least two weeks, at least four weeks, or at least eight weeks). A tyrosinase inhibitor (e.g., 3,3′-thiodipropionic acid, and/or a salt and/or an aliphatic C₁₋₂₀ alkyl ester thereof such as dilauryl thiodipropionate; or resorcinol having a C₁₋₂₀ or C₂₋₁₀ or C₄₋₈ alkyl group at the C-4 position, including n-butyl resorcinol and n-hexyl resorcinol) may also be applied prior to, after, of concurrently with the application of the glucose oxidase. A melanosome-transfer inhibitor (e.g., niacinamide or salt thereof, or melanosome-transfer inhibiting derivative thereof) may also be applied prior to, after, of concurrently with the application of the glucose oxidase.

Compositions for reducing pigmentation in human skin are provided comprising:
(i) an effective amount (e.g., from about 0.0001% to about 10% by weight, or from about 0.001% to about 5% by weight, or from about 0.01% to about 1% by weight) of glucose;
(ii) an effective amount (e.g., from about 0.00001% to about 10% by weight, or from about 0.0001% to about 1% by weight, or from about 0.005% to about 0.1% by weight) of a botanical extract (e.g., of Fabiana imbricate plant, typically from the stems and leaves) comprising an amount of glucose oxidase enzyme (e.g., at least about 1,000 units/g, or at least about 5,000 units/g, or at least about 10,000 units/g, or from about 15,000-35,000 units/g up to about 100,000 units/g or about 300,000 units/g or more) sufficient to provide at least about 0.01 activity units per gram of said composition; and
(iii) a topically acceptable vehicle (e.g., an aqueous or ethanolic serum, a water-in-oil emulsion, oil-in-water emulsion, etc., optionally comprising an emulsifier, thickener, pH adjuster, chelator, humectant, emollient, preservative, and other cosmetic adjuvants).

The compositions according to this and all other embodiments may also comprise an effective amount (e.g., from about 0.01% to about 10% by weight) of a tyrosinase inhibitor, such as 3,3′-thiodipropionic acid (TDPA), and/or a salt (e.g., sodium) and/or an aliphatic C₁₋₂₀ (C₂₋₁₈ or C₄₋₁₆ or C₆₋₁₄) alkyl ester thereof (such as dilauryl 3,3′-thiodipropionate). In some embodiments, the composition comprises from about 0.01% to about 5% by weight 3,3′-thiodipropionic acid and/or from about 0.01% to about 5% by weight dilauryl 3,3′-thiodipropionate. In other embodiments, the composition comprises from about 0.001% to about 5% or about 10% by weight of a tyrosinase inhibitor comprising resorcinol having a C₁₋₂₀ or C₂₋₁₀ or C₄₋₈ alkyl group (e.g., a straight chained alkyl group) in any ring position, for example, at the C-4 position, including n-butyl resorcinol and n-hexyl resorcinol. In some embodiments, the composition comprises an effective amount (e.g., from about 0.01% to about 10% by weight) of a melanosome-transfer inhibitor such as niacinamide or salt thereof, or melanosome-transfer inhibiting derivative thereof. In some embodiments, the compositions comprise an effective amount (e.g., from about 0.01% to about 10% by weight) of a tyrosinase inhibitor (e.g., 3,3′-thiodipropionic acid and/or dilauryl 3,3′-thiodipropionate, etc.) and an effective amount (e.g., from about 0.01% to about 10% by weight) of a melanosome-transfer inhibitor (e.g., niacinamide, etc.).

The compositions of the invention will typically include a cosmetically or dermatologically acceptable vehicle, which may be in the form of, for example, a serum, a cream, a lotion, a gel, or a stick, and may comprise an emulsion (e.g., water-in-oil, oil-in-water, water-in-silicone, silicone-in-water, polyol-in-silicone, silicone-in-polyol emulsion, etc.), or may comprise an aqueous or ethanolic vehicle, or may comprise silicone oil (e.g., cyclomethicone, dimethicone, dimethiconol, etc.), hydrocarbon oils (e.g., petrolatum, isododecane, isohexadecane, etc.), ester oils (isopropyl myristate, myristyl myristate, pentaerythritol tetraoctanoate, glyceryl trioctanoate, cetyl caprylate, etc.), fatty alcohols (e.g., behenyl alcohol, cetyl alcohol, stearyl alcohol, lauryl alcohol, etc.) or the like. The vehicle may further comprise an emulsifier (e.g., hydrogenated lecithin), surfactants (e.g., PEG-40 stearate), gelling agent, structuring agent, rheology modifier, thickener (e.g., xanthan gum, acrylates copolymers, carbopols, etc.), film former, wax (e.g., beeswax, etc.), chelators and sequestering agents (e.g., disodium EDTA), preservative (e.g., imidazolidinyl urea, methylparaben, phenoxyethanol, etc.) or the like. The compositions of the invention may optionally include additional skin benefit agents such as emollients (dimethicone oils, ester oils, or hydrocarbon oils), humectants (e.g., polyols, including propylene glycol, butylene glycol, pentylene glycol, glycerin, etc.), antioxidants (e.g., BHT, ascorbic acid, sodium ascorbate, tetrahexyldecyl ascorbate, ascorbyl palmitate, ascorbyl glucoside, beta-carotene, gamma oryzanol, glutathione, etc.), vitamins (e.g., tocopherol, tocopheryl acetate, etc.), alpha-hydroxy acids (e.g., glycolic acid), beta-hydroxy acids (e.g., salicylic acid), retinoids (e.g., retinoic acid, all-trans-retinoic acid, retinaldehyde, retinol, and retinol esters such as acetates or palmitates), other anti-aging ingredients (e.g., collagen stimulators, sodium hyaluronate), as well as additional depigmenting agents (e.g., hydroquinone, kojic acid, etc.).

In one aspect of the invention, it is believed that glucose oxidase (in the presence of glucose) and tyrosinase inhibitors, notably 3,3′-thiodipropionic acid (TDPA), or esters or salts thereof, provide a reduction in appearance of melanin that is at least 25% greater, or at least 50% greater, than achieved by the effective amount of either component alone, i.e., in the absence of the effective amount of the other component. The combination may also provide at least an approximately additive reduction in appearance of melanin, meaning that the resultant reduction in melanin is approximately the sum of the reductions achieved by the individual components alone, or the combination may provide a more than an additive reduction in the appearance of melanin, meaning that the resultant reduction in melanin is more than the sum of the reductions achieved by the individual components alone.

In another aspect of the invention, it is believed that glucose oxidase (in the presence of glucose) and melanosome transfer inhibitors, notably niacinamide or salt thereof, or melanosome-transfer inhibiting derivative thereof, provide a reduction in appearance of melanin that is at least 25% greater, or at least 50% greater, than achieved by the effective amount of either component alone, i.e., in the absence of the effective amount of the other component. The combination may also provide at least an approximately additive reduction in appearance of melanin, meaning that the resultant reduction in melanin is approximately the sum of the reductions achieved by the individual components alone, or the combination may provide a more than an additive reduction in the appearance of melanin, meaning that the resultant reduction in melanin is more than the sum of the reductions achieved by the individual components alone.

The composition may be applied once or twice daily, or more frequently, and the treatment regimen may last for as long as required to obtain the desired visible reduction in pigmentation, which may be, for example, one week, four weeks, eight weeks or longer. The compositions may be applied to human keratinous surfaces, such as skin, to treat, ameliorate, diminish, or prevent, or delay the onset of one or more of dark complexion, pigmented skin discoloration, pigmented birthmarks, hyperpigmentation, post-inflammatory hyperpigmentation, post-injury hyperpigmentation, freckles, age spots, liver spots, sun damage, tans, pigmented acne marks, scars, melasma, cholasma, after-burn scars, nail stains, yellowing of skin, or dark circles under eye.

These and other aspects of the present invention will become apparent to those skilled in the art after a reading of the following detailed description of the invention, including the illustrative embodiments and examples.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

All terms used herein are intended to have their ordinary meaning unless otherwise provided. The phrases “cosmetically acceptable,” “topically acceptable” and “dermatologically acceptable” are used interchangeably and are intended to mean that a particular component is generally regarding as safe and non-toxic at the levels employed. The term “prevent,” as used herein, includes delaying the onset of or progression of a particular sign of skin aging. The phrase “individual in need thereof” refers to a human that could benefit from improved dermal appearance or health, including males or females. In some embodiments, the individual in need thereof is a female. The term “skin” includes, without limitation, the lips, skin of the face, hands, arms, neck, scalp, and chest. As used herein, the term “consisting essentially of” is intended to limit the invention to the specified materials or steps and those that do not materially affect the basic and novel characteristics of the claimed invention, as understood from a reading of this specification.

All amounts giving are in weight percentage of the total composition (including vehicle) unless otherwise specified. Whenever a term is identified by reference to a range, the range will be understood to explicitly disclose every element thereof. As a non-limiting example, a range of 1-10% will be understood to include 1%, 2%, 3%, 4%, 5%, 6%, 7%, 8%, 9%, and 10%, and all values between 1 and 10%. Such values may be omitted herein solely for brevity.

The present invention is premised on the discovery that glucose oxidase is capable of reducing the appearance/visibility of melanin. Glucose oxidase is an oxido-reductase enzyme that catalyzes the oxidation of glucose to hydrogen peroxide (H₂O₂) and D-glucono-δ-lactone. It is contemplated that any enzyme capable of catalyzing the oxidation of glucose to yield hydrogen peroxide is suitable for the practice of the invention. Glucose oxidase may require flavin adenine dinucleotide (FAD) as a co-factor. It is believed that FAD need not be added to the compositions of the invention because it is found intrinsically in the skin cells. Alternatively, FAD may be added to the compositions of the invention in suitable amounts based on the activity levels of the glucose oxidase enzyme.

In some embodiments, the glucose oxidase enzyme is derived from a plant source, i.e., as a botanical extract. The botanical extract may comprise glucose oxidase enzyme, among other constituents, in an amount sufficient to provide at least about 100 units/g, or at least about 500 units/g, or at least about 1,000 units/g, or at least about 5,000 units/g, or at least about 10,000 units/g, or at least about 15,000 units/g of glucose oxidase enzyme. There is essentially no upper limit to the activity, and it is contemplated that activity levels up to about 100,000 units/g or even 300,000 units/g or more will be suitable. In some embodiments, the activity of the glucose oxidase ranges from about 5,000 units/g to about 100,000 units/g or from about 15,000 units/g to about 30,000 units/g. As used herein, one “unit” of glucose oxidase activity is defined as the amount of enzyme that will oxidize 1.0 μmole (micromoles) of β-D-glucose to D-gluconolactone and H₂O₂ per minute at pH 5.1 at 35° C.

The plant materials from which the glucose oxidase containing extract is obtained may be in any form including, but not limited to, the whole plant, a dried plant, a ground plant, or parts thereof, including but not limited to, seeds, needles, leaves, roots, bark, cones, stems, rhizomes, callus cells, protoplasts, flowers, nectar, fruits, and meristems, or components and/or constituents found in, or isolated from, the natural plant material, and/or portions of the plant, or any combinations thereof. In one embodiment, the extract derived from the whole plant or from a select portion of the plant, such as the flowers of the plant. In one embodiment, the extract is derived from the nectar of the plant. The plant material may be ground to small pieces or particles prior to extraction. The raw plant materials also may be dried (e.g., air-dried, oven-dried, rotary evaporated under vacuum or lyophilized) to reduce water content prior to extraction.

The glucose oxidase botanical extract may be obtained by organic solvent extraction of the plant material. The organic solvent may be polar protic, polar aprotic, or nonpolar. Non-limiting examples of organic solvents include methanol, ethanol, isopropanol, dichloromethane, chloroform, hexane (and other alkanes), xylene, and petroleum ether, and combinations thereof. An extracting machine may be used, as is well known in the field. Typically, the raw materials are pushed in the extracting machine by a thruster, which slowly moves the plant raw materials forward. Solvent (e.g., ethanol, etc.) may be added into the machine through a solvent inlet at the top of a waste discharge outlet. Due to the difference in gravity and equilibrium, the solvent flows toward the raw material inlet, soaks the materials and flows out from the opposite side of the solvent inlet. Since the plant materials and the solvent move in opposite directions against each other, the plant materials are constantly immersed in a solution that contains a low-concentration of extract. As a result of equilibrium, high yield of plant constituent(s) may be achieved by continuously extracting the plant material against the low-concentration solution.

An extraction time suitable to extract the plant constituents is used, typically between about 1-10 hours or between about 2-8 hours or between about 3-6 hours is suitable, although the invention is not limited to any particular extraction technique or conditions. The temperature of extraction may be between about 30° C.-100° C. or between about 40° C.-70° C. or between about 50° C.-60° C. The collected extract may be fine-filtered to remove debris, and may be used directly, or may be concentrated by removing solvent, for example, by evaporation under vacuum or lyophilization.

In one embodiment, the extract is obtained by aqueous extraction. Aqueous or aqueous-organic (e.g., hydro-alcoholic or water/ethanol) solvent extraction typically involves soaking the plant material in aqueous solution that is acidic, neutral, or alkaline, depending on the solubility and stability of the desired extract under acidic or alkaline (basic) conditions. For extraction under acidic conditions, an acid such as hydrochloric acid or sulfuric acid may added to water, e.g., at a concentration of about 3% (w/v). For extraction under alkaline conditions, a base such as sodium hydroxide or sodium carbonate may be added to water. The extraction time and temperature of extraction are typically similar to that used in the organic solvent extraction method described above. The extract is then typically collected and fine-filtered to remove debris. Alkaline agents (e.g., ammonia) or acidifying agents (e.g., sulfuric acid) may be added to the extract to neutralize the solution by adjusting the pH, depending on the acidity or alkalinity of the collected extract. The aqueous extract may be used directly, concentrated or dried. Alternatively, organic solvent may then be added to the neutralized solution to transfer the extract from an aqueous phase to an organic phase. Examples of such organic solvents include, but are not limited to, ethanol, isopropanol, butanol, pentanol, hexanol and xylene. The extract may be dried by a number of different means, such as, for example, air-dried, oven-dried, rotary evaporated under vacuum or lyophilized.

The extract may be further purified by chromatography (e.g., HPLC, etc.) to isolate fraction rich in glucose oxidase. The glucose-oxidase rich fraction may be further purified by diafiltration, treatment with activated charcoal and the like, and passing through a sterile filter (e.g., a 0.2 micron filter). The finished product, rich in glucose oxidase is ideally dried by lyophilization or spray drying.

In one embodiment the botanical extract comprising glucose oxidase is an extract from the plant Fabiana imbricata, a species of flowering plant in the family Solanaceae (Nightshades) native to Chile. It is also known as Pichi Pichi. It is contemplated that the glucose oxidase containing extract may be made from any of the stems, branches, leaves, flowers, or fruit or may be derived from the whole plant. In one embodiment, the glucose oxidase containing extract is obtained by extraction of the stems and leaves of Fabiana imbricata. In some embodiments, the extract is prepared by aqueous extraction of from the stems and leaves of Fabiana imbricata. In some embodiments, the extract is a solid (powder) at room temperature and can be dissolved in water at a level of 0.5% (w/w). In some embodiments, the extract of Fabiana imbricata is free of essential oils or contains less than about 5% by weight or less than about 1% by weight or less than about 0.5% by weight essential oils.

In some embodiments, the Fabiana imbricata extract may comprise glucose oxidase enzyme, among other constituents, in an amount sufficient to provide at least about 100 units/g, or at least about 500 units/g, or at least about 1,000 units/g, or at least about 5,000 units/g, or at least about 10,000 units/g, or at least about 15,000 units/g of glucose oxidase enzyme and up to about 100,000 units/g or even 300,000 units/g or more. In some embodiments, the activity of the glucose oxidase activity of the Fabiana imbricata extract ranges from about 5,000 units/g to about 100,000 units/g or from about 15,000 units/g to about 35,000 units/g.

In some embodiments, the glucose oxidase may be extracted from a microbial source, such as fungal source (e.g., Aspergillus niger). In other embodiments, the glucose oxidase is not extracted from a fungal source.

The composition of the invention will typically comprise an effective amount of the glucose oxidase containing botanical extract, such as the glucose oxidase containing extract of the Fabiana imbricata plant (e.g., an aqueous or ethanolic extract of Fabiana imbricata flowers), which will usually range from about 0.00001% to about 10% by weight, or from about 0.0001% to about 1% by weight, or from about 0.005% to about 0.1% by weight of the entire composition. Of course, the effective amount may vary depending on the glucose oxidase content and activity of the extract. In embodiment where the botanical extract, including the extract of Fabiana imbricata flowers, comprises an amount of glucose oxidase enzyme of at least about 1,000 units/g, or at least about 5,000 units/g, or at least about 10,000 units/g, or from about 15,000-30,000 units/g up to about 100,000 units/g or about 300,000 units/g or more, the effective amount will be sufficient to provide at least about 0.01 activity units/g, or at least 0.025 units/g, or at least 0.05 units/g, or at least 0.075 units/g, or at least 0.1 units/g of said composition. In some embodiments, the formulations will provide a minimum activity of glucose oxidase of at least about 0.01 activity units/g, or at least 0.025 units/g, or at least 0.05 units/g, or at least 0.075 units/g, or at least 0.1 units/g of said composition at the time of application to the skin. In some embodiments, the glucose oxidase activity of the composition does not deviate by more than about ±50%, or more than about ±25% or more than about ±10%, on storage in a sealed container (under air) at room temperate for 4 weeks, 8 weeks, 3 months, 6 months, or more.

In one embodiment, a composition of the invention comprises from about 0.0001% to about 1% by weight of a glucose oxidase containing extract of the Fabiana imbricata plant, typically obtained by aqueous or ethanolic extraction, in order to provide at least about 0.01 activity units/g of the entire composition

The glucose oxidase or glucose oxidase containing (botanical) extract may be applied to the skin in the presence of its substrate glucose. Alternatively, it may be applied in the absence of added glucose substrate and catalyze the oxidation of glucose found naturally on or in the skin. In some embodiments, the compositions of the invention include glucose oxidase and glucose. The pH of the composition may be adjusted so that catalytic activity is minimized in the bottle. The composition will typically comprise an effective amount of glucose, which may be from about 0.0001% to about 10% by weight, or from about 0.001% to about 5% by weight, or from about 0.01% to about 1% by weight of the entire composition. The amount of glucose may be in excess compared to the amount of enzyme.

The compositions may be used for reducing pigmentation and/or lightening areas of the integumentary system, including but not limited to, skin, hair, lips, and nails. The compositions are, in one embodiment, topical compositions that once applied to the biological substrate result in a lightening of the biological substrate.

In some embodiments, the composition and methods are for the treatment of hyperpigmentation, which includes eradicating, reducing, ameliorating, or reversing a degree of subject pigmentation that results from increased presence of one or more of the different types of melanin biosynthesized in skin and/or follicles and deposited in hair or skin, relative to a subject's baseline pigmentation.

In some embodiments, the composition and methods are for lightening skin, which includes eradicating, reducing, ameliorating, and/or reversing a baseline degree of subject pigmentation. Lightening skin may be measured by observing changes in Fitzpatrick scale value of a subject. The Fitzpatrick Scale (aka, Fitzpatrick skin typing test or Fitzpatrick phototyping scale) is a numerical classification schema for the color of skin, and remains a recognized tool for dermatologic research into the color of skin. The Fitzpatrick Scale measures several components, including Genetic Disposition, Reaction to Sun Exposure and Tanning Habits, and classifies skin into six types: Type I (scores 0-7) refers to white, very fair skin, freckles, typical albino skin, that always burns, never tans; Type II (scores 8-16) refers to white, fair skin, that usually burns, or tans with difficulty; Type III (scores 17-24) refers to beige, which is very common, and which sometimes suffers mild burn, gradually tans to a light brown; Type IV (scores 25-30) refers to beige skin with a brown tint, which is typical of Mediterranean Caucasian skin, and which rarely burns, tans with ease to a moderate brown; Type V (scores over 30) refers to dark brown skin which very rarely burns, tans very easily; Type VI refers to Black skin that never burns, tans very easily, and is deeply pigmented. In some embodiments of the invention, the treatments are capable of changing the treated area of skin by at least one or at least two skin type on the Fitzpatrick scale.

It is to be understood that, as used herein, the terms treating and treatment include and encompass reducing, ameliorating, improving, alleviating, and/or eliminating the dermatological effects of aging and/or environmental stress, or otherwise reducing the appearance of pigmentation in the skin. The present compositions and methods are suitable for use in treating dermatological conditions of the skin in numerous areas of the body, including, without limitation, the face, forehead, lips, neck, arms, hands, legs, knees, feet, chest, back, groin, buttocks, thighs, and the like. In one embodiment, the compositions are applied to the face, chest, arms and/or hands.

Specific benefits which may be achieved include, but are not limited to, reducing pigmentation of dark or hyperpigmented skin; reducing age spots or liver spots; reducing pigmented birthmarks, sun damage, tans, pigmented acne marks, scars; evening out or optimizing skin discoloration; decreasing the appearance of dark circles under the eyes; treating melasma, cholasma, freckles, after-burn scars, yellowing of skin, and post-injury hyperpigmentation; lightening hair on the scalp, legs, face, and other areas where whitening and color reduction are desired; and removing or reducing nail stains.

The present composition and methods of use thereof are not limited by any particular characterization of the physiological and/or chemical effects of lightening agents. Various skin lightening pathways are known and include, for example, those that occur by decreasing melanogenesis by decreasing tyrosinase activity in melanocytes as well as inhibiting melanosome maturation. However, the lightening agents used in the present compositions and methods are believed to lighten by multiple modes of action.

It may be advantageous to employ the glucose oxidase or glucose oxidase containing (botanical) extract together with other skin lightening agents such as tyrosinase inhibitors and/or melanosome transfer inhibitors.

In some embodiments, inventive compositions comprise a tyrosinase inhibitor comprising 3,3′-thiodipropionic acid (TDPA), a salt thereof, or an ester of 3,3′-thiodipropionic acid. These tyrosinase inhibitors are typically added in an effective amount of from about 0.001 % to about 10% by weight, or from about 0.005% to about 5% by weight, or from about 0.01% to about 2.5% by weight, or from about 0.05% to about 1.5% by weight, or from about 0.1 to about 1% by weight, based on the total weight of the composition. Suitable esters are disclosed in U.S. Provisional Patent Application Ser. No. 61/777,081, filed on Mar. 12, 2013, the contents of which are hereby incorporated by reference. The mono- or di-esters of TDA are typically formed by the esterification of TDPA with C₁-C₂₀ aliphatic or aromatic alcohols. More typically, the esters of TDPA are formed by the esterification of TDPA with C₆-C₁₄ straight chained alkyl alcohols, such as lauryl alcohol. In one embodiment, the composition comprises dilauryl 3,3′-thiodipropionate. In some embodiments, the composition comprises from about 0.01% to about 5% by weight 3,3′-thiodipropionic acid and/or from about 0.01% to about 5% by weight dilauryl 3,3′-thiodipropionate. In some embodiments, 3,3′-thiodipropionate or a mono-ester thereof may be present in ionized or salt form. Suitable salts may be formed by the reaction of 3,3′-thiodipropionate or its mono-ester with a base, such as, for example, a metal (sodium) hydroxide, ammonia, or an amine.

In some embodiments, the compositions of the invention will also comprise niacinamide (nicotinamide) or a derivative thereof. Suitable derivatives of niacinamide are disclosed in U.S. Patent Application Ser. No. 61/777,081, filed on Mar. 12, 2013, the disclosure of which is hereby incorporated by reference. Niacinamide or its derivative are typically added in an effective amount of from about 0.01% to about 10% by weight, or from about 0.1% to about 7.5% by weight, or from about 1% to about 5% by weight, or from about 2% to about 4% by weight by weight, based on the total weight of the composition.

In one embodiment, niacinamide (or a salt thereof) and 3,3′-thiodipropanoic acid (or a salt thereof) are each in an effective amount to lighten skin, which amount will typically range from about 0.001% to about 10% based on the total weight of the composition. Niacinamide or a salt thereof may be in an effective amount ranging from, for example, about 0.01% to about 10% (w/w), or from about 1% to about 5% (w/w) based on the total weight of the composition. The effective amount of 3,3′-thiodipropanoic acid or salts thereof may be in an effective amount of, for example, about 0.1% to about 2.5% (w/w), or from about 0.5% to about 1.5% (w/w), or about 1% (w/w) based on the total weight of the composition. The effective amount of dilauryl 3,3′-thiodipropionate or salts thereof may be in an effective amount of, for example, about 0.001% to about 2.5% (w/w), or from about 0.01% to about 1% (w/w), or from about 0.05% to about 0.5% (w/w) based on the total weight of the composition.

In some implementations of the invention, the weight ratio of the effective amount of thiodipropionic acid (TDPA), or esters or salts thereof, to the effective amount of nicotinamide, or derivative thereof, may range from about 100:1 to about 1:100, but will typically be in the range of about 50:1 to about 1:50, more typically from about 25:1 to about 1:25, or from about 15:1 to about 1:15; or from about 10:1 to about 1:10; or from about 5:1 to about 1:5; or from about 3:1 to about 1:3; or from about 2:1 to about 1:2.

The compositions can include a cosmetically or dermatologically acceptable vehicle. Such vehicles may take the form of any known in the art suitable for application to skin. The vehicle may comprise from about 50% to about 99% by weight of the composition.

The vehicle may comprise an aqueous phase, an oil phase, an alcohol, a silicone phase or mixtures thereof, and may be in the form of an emulsion. Non-limiting examples of suitable emulsions include water-in-oil emulsions, oil-in-water emulsions, silicone-in-water emulsions, water-in-silicone emulsions, polyol-in-silicone emulsions, silicone-in-polyol emulsions, polyol-in-oil emulsions, oil-in-polyol emulsions, wax-in-water emulsions, water-oil-water triple emulsions or the like. The emulsion may include an emulsifier, such as a nonionic, anionic or amphoteric surfactant, or a gelling agent.

The vehicle may comprise water; vegetable oils; mineral oils; esters such as octyl palmitate, myristyl myristate, isopropyl myristate, and isopropyl palmitate; ethers such as dicapryl ether and dimethyl isosorbide; alcohols such as ethanol and isopropanol; fatty alcohols such as cetyl alcohol, cetearyl alcohol, and stearyl alcohol; volatile silicones such as cyclomethicones, silicone oils like dimethicone, amodimethicones, and dimethiconol; hydrocarbons such as mineral oil, petrolatum, and isoparaffins such as isooctane, isododecane (IDD), isohexadecane, and isoeicosane; and (hydrogentated) polyolefins such as polyisobutene; polyols such as propylene glycol, glycerin, butylene glycol, pentylene glycol and hexylene glycol; liposomes; waxes (animal, vegetable, or synthetic); or any combinations or mixtures of the foregoing.

In one embodiment of the invention, the compositions may include additional skin actives, including but not limited to, retinoids, botanicals, keratolytic agents, desquamating agents, keratinocyte proliferation enhancers, collagenase inhibitors, elastase inhibitors, depigmenting agents, anti-inflammatory agents, steroids, anti-acne agents, antioxidants, and advanced glycation end-product (AGE) inhibitors. The amounts of these various ingredients are those conventionally used in the cosmetic field to achieve their intended purpose, and range typically from about 0.01 wt % to about 20 wt % by weight of the composition. The nature of these ingredients and their amounts must be compatible with the production of the compositions of the disclosure.

The composition may comprise additional active ingredients having anti-aging benefits, as it is contemplated that synergistic improvements may be obtained with such combinations. Exemplary anti-aging components include, without limitation, botanicals (e.g., Butea frondosa extract); phytol; retinoids (e.g., 9-cis retinoic acid, 13-cis retinoic acid, all-trans retinoic acid and derivatives thereof, phytanic acid, retinol (Vitamin A) and esters thereof, such as retinol palmitate, retinol acetate and retinol propionate, and salts thereof and others); hydroxy acids (including alpha-hydroxy acids and beta-hydroxy acids), salicylic acid and alkyl salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof, etc.), to name a few.

Exemplary retinoids include, without limitation, retinoic acid (e.g., all-trans or 13-cis), and derivatives thereof, retinaldehyde, retinol (Vitamin A) and esters thereof, such as retinol palmitate, retinol acetate and retinol propionate, and salts thereof. Particular mention may be made of retinol. When present, the retinoids will typically be included in amounts from about 0.0001% to about 5% by weight, more typically from about 0.01% to about 2.5% by weight, or from about 0.1% to about 1.0% by weight. Compositions according to this embodiment will typically include an antioxidant such as ascorbic acid and/or BHT and/or a chelating agent such as EDTA or a salt thereof (e.g., disodium EDTA).

In another embodiment, the topical compositions of the present invention may also include one or more of the following: a skin penetration enhancer; an emollient, such as isopropyl myristate, petrolatum, volatile or non-volatile silicones oils (e.g., methicone, dimethicone), ester oils, mineral oils, and fatty acid esters; a humectant, such as glycerin, hexylene glycol or caprylyl glycol; a skin plumper, such as palmitoyl oligopeptide, collagen, collagen and/or glycosaminoglycan (GAG) enhancing agents; a sunscreen, such as avobenzone or octyl methoxycinnamate; an exfoliating agent; and an antioxidant.

Suitable exfoliating agents include, for example, alpha-hydroxy acids, beta-hydroxy acids, oxa-acids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. One exemplary exfoliating agent is glycolic acid. When present, the exfoliating agent may comprise from about 0.001% to about 20% by weight of the composition.

Examples of antioxidants that may be used in the present compositions include compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives, including tocopheryl acetate; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Antioxidants may comprise, individually or collectively, from about 0.001% to about 10% (w/w), or from about 0.01% to about 5% (w/w) of the total weight of the composition.

The compositions may include one or more additional agents that combat pigmentation or hyperpigmentation, including tyrosinase inhibitors and/or melanosome transfer inhibitors. Special mention may be made of hydroquinone and the monobenzyl ether thereof; hydroquinone-beta-D-glucopyranoside; retinoids (e.g., retinoic acid); tretinoin; azelaic acid; Kojic acid (5-hydroxy-4-pyran-4-one-2-methyl); Mequinol (4-hydroxyanisole); soy protein and other serine protease inhibitors; paper mulberry extract; Glabridin (licorice extract); Arctostaphylos patula and Arctostaphylos viscida extracts; Glycyrrhiza glabra and its derivatives; Chlorella vulgaris extract; Magnesium-L-ascorbyl-2-phosphate (MAP); 4-Isopropylcatechol; Aleosin; N-acetyl-4-S-cysteaminylphenol and N-propionyl-4-S-cysteaminylphenol; N-acetyl glucosamine; and Tranexamic acid (trans-4-aminomethylcyclohexanecarboxylic acid); arbutin, bearberry extract, ascorbic acid and/or its derivatives, perilla extract (e.g., in U.S. Pat. No. 5,980,904 and Japanese Publications Nos. 07025742, 07187989, 10265322, 2001163759, and 2001181173, incorporated herein by reference), coconut fruit extract (Japanese Patent No. 2896815B2, incorporated by reference herein), coconut water, and calcium influx inhibitors, to name a few. Any of the tyrosine inhibitors disclosed in KR 2005095167; JP 2003252743; and JP 61260009, incorporated by reference herein, may be included, in some embodiments. In another embodiment of the invention, the compositions may include any of the following ingredients, alone or in combination: nilopala; patanga; chandana; ushira; manjshta; kumkuma; laksa; padmakesara; padmaka; yashtimadhu; ajakshira; ksheera; nyagrodhapada; and/or lodhra.

Other skin lighteners include extracts of Butea frondosa, Naringi crenulata, Stenoloma chusana, Azadirachta indica, Glycyrrhiza glabra linn., Morinda citrifolia, tomato glycolipid, ascorbyl glucoside, vitamin C, retinol and/or its derivatives, rumex crispus extract, milk proteins including hydrolyzed milk proteins, N,N,S-tris(carboxymethyl)cysteamine, oleanolic acids, placenta extract, saxifragia sarmentosa, juniperic acid, ligusticum chiangxiong hort., asmunda japonica thunb., stellaria medica (L.) cyr., sedum sarmentosum bunge, ligusticum lucidum Ait., ilex purpurea hassk, emblica, apigenin, ascorbyl palmitol, carruba C. borealis s, hesperitin, inabata C. borealis, isoliquirtigenin, kaempherol-7-neohesperidose, L-mimosine, luteolin, oil-soluble licorice extract P-T(40), oxa acid, phenyl isothiocyanate, cococin, silymarin, T4CA, teterahydro curcumin, unitrienol, ursolic-oleanolic acid, UVA/URSI, or any combinations thereof. In one embodiment, the compositions comprise Soybean Trypsin Inhibitor (STI).

Other additives include: vitamins, such as tocopherol and ascorbic acid; vitamin derivatives such as ascorbyl monopalmitate, tocopheryl acetate, and Vitamin E palmitate; thickeners such as hydroxyalkyl cellulose, carboxymethylcellulose, carbombers, and vegetable gums such as xanthan gum; gelling agents, such as ester-terminated polyester amides; structuring agents; metal chelating agents such as EDTA or salts thereof; pigments; colorants; and pH adjusters (citric acid, ethanolamine, sodium hydroxide, etc.). The composition may optionally comprise other components known to those skilled in the art including, but not limited to, film formers, moisturizers, minerals, viscosity and/or rheology modifiers, anti-acne agents, insect repellents, skin cooling compounds, skin protectants, lubricants, fragrances, preservatives, stabilizers, and mixtures thereof. In addition to the foregoing, the cosmetic compositions of the invention may contain any other compound for the treatment of skin disorders.

In a specific embodiment, the composition may comprise at least one additional botanical, such as, for example, a botanical extract, an essential oil, or the plant itself. Suitable botanicals include, without limitation, extracts from Abies pindrow, Acacia catechu, Anogeissus latifolia, Asmunda japonica, Azadirachta indica, Butea frondosa, Butea monosperma, Cedrus deodara, Emblica officinalis, Ficus benghalensis, Glycyrrhiza glabra, Ilex purpurea Hassk, Inula racemosa, Ligusticum chuangxiong, Ligusticum lucidum, Mallotus philippinensis, Mimusops elengi, Morinda citrifolia, Moringa oleifera, Naringi crenulata, Nerium indicum, Psoralea corylifolia, Stenoloma chusana, Terminalia bellerica, tomato glycolipid and mixtures thereof. In one embodiment, the composition comprises a yeast extract, such as an aqueous yeast extract. In another embodiment, the composition comprises algae (Phaedectylum tricornutum) extract. In another embodiment, the composition comprises an aqueous soybean extract. In another embodiment, the composition comprises algae (Phaedectylum tricornutum) extract. In yet another embodiment, the composition comprises an kudzu (Pueraria lobata) extract. Such additional botanicals and extracts may be present, individually or collectively, in an amount from about 0.0001% to about 25% by weight of the composition.

In addition, the compositions contemplated by this disclosure can include one or more compatible cosmetically acceptable adjuvants commonly used and known by the skilled practitioner, such as colorants, pearls, chromalites, micas, pigments, dyes, fragrances, emollients, humectants, preservatives, vitamins, chelators, thickeners, anesthetics, anti-allergenics, antifungals, antimicrobials, other anti-inflammatory agents, antioxidants, antiseptics, depigmenting agents, film formers, insect repellents, pharmaceutical agents, photostabilizing agents, sunscreens, stabilizers, surfactants, thickeners, viscosity modifiers, and botanicals. The topical compositions of the present disclosure may also include a skin penetration enhancer, a surface smoother, a skin plumper, an optical diffuser, an exfoliation promoter, and an antioxidant. Details with respect to these and other suitable cosmetic ingredients can be found in the “International Cosmetic Ingredient Dictionary and Handbook,” 10th Edition (2004), published by the Cosmetic, Toiletry, and Fragrance Association (CTFA), at pp. 2177-2299, which is herein incorporated by reference in its entirety. The amounts of these various substances are those that are conventionally used in the cosmetic or pharmaceutical fields, for example, they can constitute from about 0.01% to about 20% of the total weight of the composition.

A sunscreen may be included to protect the skin from damaging ultraviolet rays. In an illustrative embodiment of the present disclosure, the sunscreen provides both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, octocrylene, titanium dioxide, zinc oxide, or any mixtures thereof. The sunscreen may be present from about 1 wt % to about 30 wt % of the total weight of the composition.

The composition may be formulated in a variety of product forms, such as, for example, an emulsion, lotion, cream, serum, spray, aerosol, cake, ointment, essence, gel, paste, patch, pencil, towelette, mask, stick, foam, elixir, concentrate, and the like, particularly for topical administration. The composition is typically formulated as an emulsion, lotion, cream, ointment, serum or gel. The compositions can be formulated into liposomes which can comprise other additives or substances, and/or which can be modified to more specifically reach or remain at a site following administration.

The composition may include an emulsifier, typically in an amount from about 0.001-10% by weight. Non-limiting emulsifiers include nonionic, anionic, amphoteric, and zwitterionic surface active agents. Suitable emulsifiers include but are not limited to emulsifying waxes, emulsifying polyhydric alcohols, polyether polyols, polyethers, mono- or di-ester of polyols, ethylene glycol mono-stearates, glycerin mono-stearates, glycerin di-stearates, silicone polyols, soya sterols, fatty alcohols such as cetyl alcohol, acrylates, fatty acids such as stearic acid, fatty acid salts, and mixtures thereof. Other specific emulsifiers that can be used in the composition of the present invention include, but are not limited to, one or more of the following: C10-30 alkyl acrylate crosspolymer; Dimethicone PEG-7 isostearate, sorbitan esters; polyglyceryl-3-diisostearate; sorbitan monostearate, sorbitan tristearate, sorbitan sesquioleate, sorbitan monooleate; glycerol esters such as glycerol monostearate and glycerol monooleate; polyoxyethylene phenols such as polyoxyethylene octyl phenol and polyoxyethylene nonyl phenol; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, to name a few. Additional emulsifiers are provided in the INCI Ingredient Dictionary and Handbook 13th Edition 2010, the disclosure of which is hereby incorporated by reference. In some embodiments, the emulsifier may comprise hydrogenated lecithin, steareth-2, and/or methyl glucosides (e.g., POE (20M) Methyl Glucose Ether), and the like. These emulsifiers typically will be present in the composition in an amount from about 0.001% to about 10% by weight, in particular in an amount from about 0.01% to about 5% by weight, and more preferably, from about 0.1% to about 3% by weight.

In one embodiment, the topical composition will have a pH range from 1 to 13, with a pH in the range of from 2 to 12 being typical. In some embodiment, the composition will have a pH in the range of from 3.5 to 7 or from 7-10.5. In some embodiments, the pH will be in the range of 3-4, or 4-5, or 5-6, or 6-7, or 7-8, or 8-9, or 9-10, or 10-11, or 11-12. Suitable pH adjusters such as sodium hydroxide, citric acid and triethanolamine may be added to bring the pH within the desired range.

Another embodiment of the present disclosure is directed to the delivery of the described compositions by the use of targeted delivery systems, for example, liposomes, microspheres (see, e.g., U.S. Pat. No. 5,770,222 to Unger et al.), and the like, so that the components and/or active constituents can more readily reach and affect the subcutaneous layer of the area of application, e.g., face or neck, or the other area of the skin.

In certain embodiments, the cosmetic compositions described herein can be used to treat and/or prevent hyper-pigmentation of skin and/or of the hair, for example, to lighten skin or hair. In some embodiments, a composition is topically applied to the skin or hair, for example to an area of hyper-pigmented skin or hair. Hyper-pigmentation includes any coloration of an individual's skin or hair that is darker than desired by the individual and that is caused by melanocytes. Such unwanted pigmentation may also be called discoloration. Hyper-pigmented areas of the skin include areas of discrete or mottled hyper-pigmentation. Areas of discrete hyper-pigmentation can be distinct, uniform areas of darker color and may appear as brown spots or freckles on the skin, including marks commonly called pigment spots or “age spots.” Areas of mottled hyper-pigmentation of the skin can be dark blotches that are larger and more irregular in size and shape than areas of discrete pigmentation. Areas of hyper-pigmentation also include areas of tanned skin, for example, skin tanned due to UV exposure. Hyper-pigmented hair includes any shade of hair that is darker than desired.

Skin hyper-pigmentation may be caused by any number of factors, including, for example, genetics, UV or sun exposure, age, scarring, or discoloration due to skin injury, including lacerations, burns, sunburn, acne, or other dermatological conditions, and the like. For example, skin hyper-pigmented areas include melasmic patches. Melasma is a common skin disorder involving facial skin discoloration, one embodiment prevalent in pregnant women, where it is called chloasma faciei or chloasma. Melasmic (or chloasmic) patches may appear as dark brown, irregular patches on the face, on the upper cheeks, nose, lips, upper lip, and forehead. The patches often develop gradually over time and generally do not itch or otherwise hurt, but may negatively affect an individual's appearance Skin hyper-pigmentation also refers to areas under the arm, e.g., that have become or are becoming darker than desired.

Skin hyper-pigmentation may or may not include areas under an individual's eyes that are darker than desired by the individual, commonly referred to as “under eye dark circles” or “dark circles.” Dark circles are usually round, uniform areas of pigmentation beneath each eye, which may be caused by heredity, allergies, tiredness, or other causes. In one embodiment, the compositions are topically applied for the treatment of under eye dark circles. However, treatment of hyper-pigmentation, in some embodiments, excludes treating discoloration and/or bagginess in facial skin below the eyes because such pigmentation may entail an unrelated etiology to other hyperpigmentation conditions. Hyper-pigmented skin may also include skin in the axillary (i.e., underarm) region.

Treating hyper-pigmentation or hyper-pigmented skin/hair refers to eradicating, reducing, ameliorating, or reversing one or more of the unwanted features associated with hyper-pigmentation, such as producing a perceptible lightening of the skin or hair in the affected area. Lightening hyper-pigmented areas of the skin may be desirable, in one embodiment, in diminishing age spots; lightening a suntan; evening or optimizing skin tones, e.g., in areas of mottled hyper-pigmentation; in treating melasmic and chloasmic patches, freckles, after-burn scars, and post-injury hyper-pigmentation. Preventing hyper-pigmentation or hyper-pigmented skin refers to affording skin, not yet affected by hyper-pigmentation, a benefit that serves to avoid, delay, forestall, or minimize one or more unwanted features associated with skin hyper-pigmentation, such as reducing the darkness or size of hyper-pigmented areas that eventually develop.

The inventive compositions are capable of treating and/or preventing hyper-pigmented skin and can be referred to as “skin lighteners.” When used for lightening hair, they can be referred to “hair lighteners.” In one embodiment, the compositions of the present invention are usable to lighten hair in a non-bleaching manner; that is, by suppressing the formation and/or transportation of melanin out of follicular melanocytes, rather than by bleaching the hair itself. In one embodiment, the hair lightened by the instant invention includes facial hair (e.g., hair above the upper lip) and body hair (e.g., arms and legs), as opposed to scalp hair. In one embodiment, the hair lightener is applied to facial hair located on the upper lip.

The compositions are applied to the skin for a period of time sufficient to diminish the appearance of melanin in the skin. The compositions may be applied topically once, twice, or more daily. The treatment may be for a period of one week, two weeks, four weeks, eight weeks, or more. In one embodiment, the compositions of the invention will be applied to the skin in an amount from about 0.001 to about 100 mg/cm², more typically from about 0.01 to about 20 mg/cm², or from about 0.1 to about 10 mg/cm².

The present inventive compositions provide for products, especially skin care and cosmetic products that lighten skin in need thereof. Skin in need thereof includes, but is not limited to, dark complexions, hyperpigmented skin, age spots, liver spots, discolored or uneven skin, dark circles under the eyes for example, skin having melasma, cholasma, freckles, after-burn scars, post-injury hyperpigmented skin, skin, scalp, legs, face, and other areas where whitening or color reduction are desired, yellowed skin, stained nails, and the like.

One embodiment of the invention relates to methods of applying an effective amount of the lightening composition described herein, to lighten an affected area of the skin as used herein. The lightening composition is, in one embodiment, topical and applied once or twice daily, where the affected area of the skin that is in need of lightening includes, but is not limited to, the face, neck, hands, arms, legs, feet, thighs, hair, scalp, and overall body. The lightening composition may remain on the affected area in need of lightening or may be rinsed off or otherwise removed depending on the application. In order to maintain the desired lightening effect, the protocol should be continued for as long as the lightening effect is desired. Once the application of the lightening composition is discontinued, the desired lightening effect will also diminish.

In a related embodiment, the compositions of the invention are applied to human skin to reduce sebum production or improve the appearance of skin affected by cellulite, and/or reduce unwanted lipogenesis or increase lipolysis. In this embodiment, the compositions can be formulated in cosmetically acceptable vehicles (as described herein) and may include one or more additional agents such as anti-acne ingredients (e.g., salicylic acid, benzoyl peroxide and other peroxides, sulfur, retinoids, etc.) in the case of a facial composition, or, in the case of a cellulite treatment, the formulation may comprise any ingredients suitable for treatment of cellulite, including without limitation, perilla oil and other unsaturated fatty oils and omega-3 fatty acids such as alpha-linolenic acid; caffeine; theophylline; xanthines; retinoids (e.g., retinol); and the like. A cellulite treatment according to the invention will typically be applied topically to skin suffering from cellulite, including skin of the buttocks and thighs for a period of time sufficient to improve the appearance thereof, including for example, daily treatment for at least four weeks, at least eight weeks, at least twelve weeks, or longer.

In another embodiment, the compositions are applied to skin affected by acne (e.g., at least once daily) for a time sufficient to achieve a reduction in the appearance of acne. It is contemplated that the release of hydrogen peroxide by the action of glucose oxidase on glucose will kill or reduce the number of bacteria that promote acne (e.g., Propionibacterium acnes). Compositions according this embodiment may further include additional anti-acne agents such as sulfur, retinoids (e.g., retinol), salicylic acid and derivatives (e.g., di-salicylic acid, and C₁₋₂₀ alkyl derivatives, including esters and ethers), hydroxynapthoic acids, and derivatives, etc., and peroxides, such as benzoyl peroxide.

In some embodiments, compositions will be used to treat signs of chronological and environmental aging, including reducing the severity of fine lines or wrinkles, treating thin skin, which includes thickening skin that has already thinned, and treating sagging skin. The compounds are often in combination with retinol in this embodiment. The composition will typically be applied to the skin one, two, or three times daily for as long as is necessary to achieve desired results. The treatment regimen may comprise daily application for at least one week, at least two weeks, at least four weeks, at least eight weeks, or at least twelve weeks or more. Chronic treatment regimens are also contemplated. The effect of a composition on the formation or appearance of fine lines and wrinkles can be evaluated qualitatively, e.g., by visual inspection, or quantitatively, e.g., by microscopic or computer assisted measurements of wrinkle morphology (e.g., the number, depth, length, area, volume and/or width of wrinkles per unit area of skin).

In other embodiments, the compositions are topically applied to the skin to achieve an aesthetic improvement in skin. The aesthetic improvement of human skin may be an improvement of any attribute or characteristic of skin, including without limitation:

(a) treatment, reduction, and/or prevention of fine lines or wrinkles;

(b) reduction of skin pore size;

(c) improvement in skin thickness, plumpness, and/or tautness;

(d) improvement in skin smoothness, suppleness and/or softness;

(e) improvement in skin tone, radiance, and/or clarity;

(f) improvement in procollagen, and/or collagen production;

(g) improvement in maintenance and remodeling of elastin;

(h) improvement in skin texture and/or promotion of retexturization;

(i) improvement in skin barrier repair and/or function;

(j) improvement in appearance of skin contours;

(k) restoration of skin luster and/or brightness;

(l) replenishment of essential nutrients and/or constituents in the skin;

(m) improvement of skin appearance decreased by aging and/or menopause;

(n) improvement in skin moisturization;

(o) increase in skin elasticity and/or resiliency;

(p) treatment, reduction, and/or prevention of skin sagging;

(q) improvement in skin firmness;

(r) reduction of pigment spots and/or mottled skin;

(s) improvement of optical properties of skin by light diffraction or reflection;

(t) improvement in skin fairness; and

(u) treatment of acne.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, §201(i).

### EXAMPLES

The following example illustrates a specific aspect of the instant description. The example should not be construed as limiting, as the example merely provides specific understanding and practice of the embodiments and its various aspects.

### Example 1

Fabiana imbricata extract (having between 15,000 and 30,000 units/g of glucose oxidase activity) was examined for its ability to lighten melanin in an in vitro test. A solution of synthetic melanin at a concentration of 0.025% in dimethyl sulfoxide was prepared. 1 ml was added to each well of a 24-well plate. A 1% stock solution of Fabiana imbricata extract was prepared in phosphate-buffered saline and diluted serially at a ratio of 1:2 yielding 0.5%, 0.25% and 0.125% solutions of Fabiana imbricata extract. Solutions of glucose were prepared similarly. 10 microliters of each concentration of Fabiana imbricata extract was added along with 10 microliters of glucose to 1 ml of the melanin solution in each well of the microplate. Each concentration was tested in triplicate. Three wells received the phosphate-buffered saline as vehicle control. Hydrogen peroxide at 0.3, 0.03 and 0.003% was added to 3 wells each as positive control. The microplate was incubated at room temperature and treatments described above were repeated for a total of ten times over two weeks. At the end of the incubation period, melanin absorbance was measured at 405 nm using a spectrophotometer. Results are summarized in the table below as percent of melanin absorbance relative to vehicle control. A lower absorbance indicates lightening of melanin.

TABLE 1

| Sample | % Relative Absorbance |
|---|---|
| Vehicle | 100 |
| 0.01% Fabiana extract | 66.6 |
| 0.005% Fabiana extract | 67.6 |
| 0.0025% Fabiana extract | 69.0 |
| 0.00125% Fabiana extract | 69.7 |
| 0.3% H₂O₂ | 30.6 |
| 0.03% H₂O₂ | 70.6 |
| 0.003% H₂O₂ | 87.5 |

### Examples 2-4

Table 2 provides four skin lightening formulations comprising a glucose oxidase containing extract of the leaves and stems of the Fabiana imbricate plant.

TABLE 2

| Ingredient | Example 1 | Example 2 | Example 3 | Example 4 |
|---|---|---|---|---|
| Demineralized water | q.s | q.s | q.s | q.s. |
| Glycol | 5.0000 | 5.0000 | 5.0000 | q.s. |
| Carbopol 934 | 0.3500 | — | — | 0.10-0.20 |
| Acrylates/C10-30 Alkyl Acrylate | 0.10-0.20 | 0.10-0.20 | 0.10-0.20 | 0.10-0.20 |
| Crosspolymer | | | | |
| Xanthan gum | 0.1500 | — | — | — |
| Niacinamide | 0.1-10 | 0.1-10 | 0.1-10 | 0.1-10 |
| Thiodipropionic acid | 0.1-10 | — | — | 0.1-10 |
| Disodium EDTA | 0.1-10 | 0.1-10 | 0.1-10 | 0.1-10 |
| Methylparaben | 0.2000 | — | 0.0000-0.2000 | 0.0000-0.2000 |
| Ethoxydiglycol | 1.000-2.000 | 1.000-2.000 | — | 1.000-2.000 |
| Steareth-2 | 0.1-1.0 | 0.1-1.0 | — | 0.1-1.0 |
| PEG-40 Stearate | 0.1-10 | 0.1-10 | 0.1-10 | 0.1-10 |
| Dilauryl thiodipropionate | 0.1-10 | 0.1-10 | 0.1-10 | 0.1-10 |
| Tetrahexyldecyl ascorbate | 0.0010-10 | 0.0010-10 | 0.0010-10 | 0.0010-10 |
| Anti-aging actives | 0.0001-2 | 0.0001-2 | 0.0001-2 | 0.0001-2 |
| Isododecane | 2.0000 | — | — | — |
| Dimethicone/Dimeth. | 1.0000 | 2.0000 | — | 1.0000-5.0000 |
| Crosspolymer | | | | |
| Silicone fluid | 1.0000 | — | 3.0000 | — |
| Silica shells | 0.2000 | 0.2500 | — | 0.2500 |
| Alcohol | 4.3000 | — | — | — |
| Phenoxyethanol | 0.3000 | 0.5000 | 0.5000 | 0.5000 |
| Ascorbyl glucoside | 1.0000 | 1.0000 | 1.0000 | 1.0000 |
| Sodium hydroxide solution 50% | 1.9200 | 0.5100 | 0.7000 | 0.10000-1.0000 |
| Yeast extract | 0.3000 | 0.1000 | 0.1000 | 0.1000 |
| Yeast polysaccharides (fermented | 0.3000 | 0.1000 | 0.1000 | 0.1000 |
| w/Clintonia borealis extract) | | | | |
| Glucose | 0.001-1 | 0.001-1 | 0.001-1 | 0.001-1 |
| Hydrolyzed rice | 0.0100-1 | 0.0100-1 | 0.0100-1 | 0.0100-1 |
| protein/hydrolyzed wheat protein | | | | |
| Glutathione | 0.0010-1 | 0.0010-1 | 0.0010-1 | 0.0010-1 |
| Fabiana imbricata leaf/stem | 0.0001-1 | 0.0001-1 | 0.0001-1 | 0.0001-1 |
| extract | | | | |
| Colorant | 0.1000-1 | 0.1000-1 | 0.1000-1 | 0.1000-1 |
| Carbopol 940 | 0-1 | 0-1 | 0-1 | 0-1 |
| Glycerin | 0-10 | 0-10 | 0-10 | 0-10 |
| Pentaerythritol tetraoctanoate | 0-2 | 0-2 | 0-2 | 0-2 |
| POE (20M) Methyl Glucose Ether | 0-1 | 0-1 | 0-1 | 0-1 |
| Antioxidant(s) | — | 0.0001-5% | 0.0001-5% | 0-5 |
| Caprylic CapricTriglycerides | — | 0.0200 | 0.0200 | 0-1 |
| Dimethicone/Dimethiconol 87/13% | — | 1.2500 | — | 0-2 |
| Isohexadecane | — | 4.0000 | — | 0-5 |
| Imidazolidinyl urea | — | 0.5000 | — | 0-1 |
| Soybean (gly. soja) extract | — | 0.1000 | 0.1000 | 0-1 |
| Spherical Silica (2-20 microns) | — | 1.3000 | — | 0-5 |
| Spherical PMMA | — | 2.4000 | — | 0-5 |
| Sodium Hyaluronate | — | — | 0.0500 | 0-1 |
| Cetyl Caprylate | — | — | 4.0000 | 0-5 |
| Glyceryl Ttrioctanoate | — | — | 4.0000 | 0-5 |
| Beeswax | — | — | 0.5000 | 0-1 |
| Behenyl alcohol | — | — | 0.2500 | 0-1 |
| Cetyl/Stearyl alcohol (60/40) | — | — | 0.2500 | 0-1 |
| Soya lecithin/cholesterol blend | — | — | 0.2500 | 0-1 |
| Hydrogenated Lecithin | — | — | 0.2500 | 0-1 |
| Dimethyl Polysiloxane | — | — | 0.6000 | 0-1 |
| Hydroxyethyl Acrylate/Sod | — | — | 2.0000 | 0-5 |
| Acryloyldimethyl Copolymer | | | | |
| Taurate/Isohexadecane/Polysorbate | | | | |
| 60 Phytol | | | | |

### Example 5

A 3D Epidermal skin equivalent model consisting of keratinocytes and dark pigmented melanocytes (Melanoderm, MEL-300B; MatTek Corporation, Ashland, Mass.) was used to assess reduction in pigmentation by topical application of actives. The melanoderm tissues were cultured in 6-well plates and were incubated at 37° C. and 5% CO₂ humidified incubator. Melanoderm tissues were treated with either vehicle or a blend of actives including Fabiana extract for 14 days. The actives blend consisted of Fabiana extract, glucose, thiodipropionic acid, ascorbyl glucoside, niacinamide, yeast extract, rice extract, glutathione and phytol. The vehicle consisted of DMSO, ethanol and water. On the last day of experiment, tissues were collected for assay of melanin content. Briefly, tissues were digested using Solvable™ (MatTek Coproration, Ashland, Mass.), incubating overnight at 60° C. Each sample was vortexed vigorously and checked to see if the tissue was completely digested. The extracted samples were centrifuged at 13,00 rpm for 5 minutes and absorbance was measured in triplicate using a microplate spectrophotometer at 490 nm.

The percentage relative absorbance of the tissues treated with the active blend including Fabiana extract was 16.5% lower than that of the tissues treated with the vehicle. This reduction was statistically significant at p<0.05.

The contents of all patents, patent applications, published articles, abstracts, books, reference manuals and abstracts, as cited herein are hereby incorporated by reference in their entireties to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

1. A method for reducing pigmentation in human skin comprising topically applying to said skin a composition comprising a botanical extract of Fabiana imbricata comprised of glucose oxidase enzyme.

2. The method of claim 1, wherein the botanical extract of Fabiana imbricata comprises at least about 1,000 units/g of glucose oxidase enzyme.

3. The method of claim 1, wherein the composition is further comprised of niacinamide.

4. The method of claim 1, wherein the composition is further comprised of from about 0.001% to about 10% by weight glucose.

5. The method of claim 1, wherein the composition is further comprised of from about 0.01% to about 10% by weight tyrosinase inhibitor comprising 3,3′-thiodipropionic acid, and/or a salt and/or aliphatic C₁₋₂₀ alkyl ester thereof, or resorcinol or a derivative having a C1-20 n-alkyl group at the C-4 position.

6. A composition for reducing pigmentation in human skin comprising:
(i) from about 0.001% to about 10% by weight glucose;
(ii) from about 0.00001 to about 1% by weight of a botanical extract comprising an amount of glucose oxidase enzyme sufficient to provide at least about 0.01 activity units per gram of said composition; and
(iii) a topically acceptable vehicle.

7. The composition of claim 6, wherein the botanical extract comprises a botanical extract of Fabiana imbricate.

8. The composition of claim 6, further comprising from about 0.01% to about 10% by weight tyrosinase inhibitor comprising 3,3′-thiodipropionic acid, and/or a salt and/or aliphatic C₁₋₂₀ alkyl ester thereof, or resorcinol or a derivative having a C₁₋₂₀ n-alkyl group at the C-4 position.

9. The composition of claim 6, further comprising from about 0.01% to about 10% by weight of a melanosome-transfer inhibitor comprising nicotinamide, derivative or salt thereof.

10. A method for reducing hyperpigmentation in human skin comprising topically applying a botanical extract having at least about 1,000 units/g of glucose oxidase enzyme to an area of skin affected by hyperpigmentation.
